(19) United States
(12) Patent Application Publication
(10) Pub. No.: US 2015/0190410 A1
(43) Pub. Date: Jul. 9, 2015

(54) USE OF CREATINE AND PHOSPHOCREATINE IN THE TREATMENT OF EPILEPSY AND ALCOHOL ADDICTION

(71) Applicant: Dignity Health, Phoenix, AZ (US)

(72) Inventor: Chuanzhe Song, Avondale, AZ (US)

(73) Assignee: Dignity Health, Phoenix, AZ (US)

(21) Appl. No.: 14/414,389

(22) PCT Filed: Jul. 16, 2013
(86) PCT No.: PCT/US2013/050759

§ 371 Date: Jan. 12, 2015

Related U.S. Application Data
(60) Provisional application No. 61/672,174, filed on Jul. 16, 2012.
(60) Provisional application No. 61/672,577, filed on Jul. 17, 2012.

(51) Int. Cl. A61K 31/66; A61K 31/197

(52) U.S. Cl. CPC A61K 31/66; A61K 31/197

(57) ABSTRACT

The present invention relates to neurological disorders and treatment of epilepsy, seizures and alcoholism. In one embodiment, the present invention provides a method of treating the frequency of epileptic seizures in an subject by administering a therapeutically effective dosage of a composition comprising creatine and/or phosphocreatine. In another embodiment, the present invention provides a method of treating alcoholism by administering a composition comprising creatine and/or phosphocreatine.

### BACKGROUND

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

GABA, or gamma-aminobutyric acid, is a major inhibitory neurotransmitter within the brain, and may have an important role in many neurological diseases and disorders, including both epilepsy and alcoholism. Epilepsy is the second most common neurological disorder characterized by spontaneous recurrence of unprovoked seizures. The disorder affects approximately 1 to 3% of the population, and the prevalence of epilepsy at any point in time is between 5 and 9 individuals per 1000. The incidence of newly diagnosed epilepsy is between 30 to 50 individuals per 100K person per year. However, despite its prevalence and damaging effects, there still remains a lack of suitable treatment. Current antiepileptic drugs have several problems. Existing antiepileptic drugs have many dose-related side effects such as depression, psychosis, suicidal thoughts, liver problems, diplopia, nausea, dizziness, headache, fatigue, tiredness, ataxia, and chronic side effects such as changes in the way a person looks or fells, damage to their bones or tendons, and infertility. In addition, existing antiepileptic drugs are only effective for up to 60˜70% of patients. Additionally, while currently the Ketogenic diet may be effective in treating epilepsy for some individuals, the diet is not very well suited for adults due to its demanding dietary regiment. Similarly, alcoholism is also a severe disease and problem that affects countless individuals and has a significant detrimental impact, both economic and social. The disease of alcoholism is characterized by an increased tolerance and physical dependence on alcohol. Long term alcoholism can cause symptoms such as cirrhosis, pancreatitis, epilepsy, dementia, heart disease and nutritional deficiencies, and can ultimately prove to be fatal. Thus, there is a need in the art for novel and effective treatments of alcoholism, epilepsy and seizures in general.

### SUMMARY OF THE INVENTION

Various embodiments include a method of treating epileptic seizures in an subject, comprising providing a composition comprising a therapeutically effective dosage of creatine, cyclocreatine, phosphocreatine, or a derivative, analog, salt, and/or pharmaceutical equivalent thereof, and administering the composition to the subject. In another embodiment, the therapeutically effective dosage is between 5 mg/kg to 15 mg/kg. In another embodiment, the therapeutically effective dosage is between 15 mg/kg to 50 mg/kg. In another embodiment, the therapeutically effective dosage is between 50 mg/kg to 100 mg/kg. In another embodiment, the therapeutically effective dosage is between 100 mg/kg to 500 mg/kg. In another embodiment, the therapeutically effective dosage is between 500 mg/kg to 1000 mg/kg. In another embodiment, the therapeutically effective dosage is between 1000 mg/kg and 1500 mg/kg. In another embodiment, the composition is administered twice daily. In another embodiment, the composition is administered once a day. In another embodiment, the composition is administered orally. In another embodiment, the subject is a human. In another embodiment, the subject is a rodent. In another embodiment, the subject has previously experienced an epileptic seizure. In another embodiment, the subject has previously experienced an epileptic seizure less than 3 days before treatment. In another embodiment, the subject has previously experienced an epileptic seizure between 3 to 7 days before treatment. In another embodiment, the subject has previously experienced an epileptic seizure between 7 days to 30 days before treatment. In another embodiment, the subject has previously experienced an epileptic seizure more than 30 days before treatment. In another embodiment, the composition is administered in conjunction with additional seizure treatments. In another embodiment, administering the composition results in a decrease in frequency of epileptic seizures in the subject. In another embodiment, administering the composition results in a decrease in duration of epileptic seizures in the subject.

Other embodiments include a method of treating alcohol addiction in a subject, comprising providing a composition comprising a therapeutically effective dosage of creatine, cyclocreatine, phosphocreatine, or a derivative, analog, salt, and/or pharmaceutical equivalent thereof, and administering the composition to the subject. In another embodiment, the therapeutically effective dosage is between 5 mg/kg to 15 mg/kg. In another embodiment, the therapeutically effective dosage is between 15 mg/kg to 50 mg/kg. In another embodiment, the therapeutically effective dosage is between 50 mg/kg to 100 mg/kg. In another embodiment, the therapeutically effective dosage is between 100 mg/kg to 500 mg/kg. In another embodiment, the therapeutically effective dosage is between 500 mg/kg to 1000 mg/kg. In another embodiment, the therapeutically effective dosage is between 1000 mg/kg and 1500 mg/kg. In another embodiment, the composition is administered twice daily. In another embodiment, the composition is administered once a day. In another embodiment, the composition is administered orally. In another embodiment, the subject is a human. In another embodiment, the subject is a rodent.

Other embodiments include a pharmaceutical composition, comprising a therapeutically effective dosage of creatine, cyclocreatine, phosphocreatine, or a derivative, analog, salt, and/or pharmaceutical equivalent thereof, and a pharmaceutically acceptable carrier. In another embodiment, the therapeutically effective dosage is about 30 mg/kg. In another embodiment, the therapeutically effective dosage is about 100 mg/kg. In another embodiment, the therapeutically effective dosage is about 1000 mg/kg.

Other features and advantages of the invention will become apparent from the following detailed description, taken in conjunction with the accompanying drawings, which illustrate, by way of example, various embodiments of the invention.

### BRIEF DESCRIPTION OF FIGURES

FIG. 1 depicts, in accordance with an embodiment herein, results of twice daily administration of 30 mg/kg creatine for epilepsy treatment.

FIG. 2 depicts, in accordance with an embodiment herein, creatine as a partial negative allosteric modulator of extra-synaptic GABAA receptors.

FIG. 3 depicts, in accordance with an embodiment herein, utilizing whole cell patch clamp on primary cultured neuron (rat cortex) to confirm creatine's effect on extra-synaptic GABA_{A} receptors.

FIG. 4 depicts, in accordance with an embodiment herein, therapeutic treatment with creatine analog cyclocreatine.

FIG. 5 depicts, in accordance with an embodiment herein, a chart of 6 days, over one month of time, with data points for both midnight and noon for each day. As evidenced by entries for the 16^{th}, seizures dramatically decreased in conjunction with treatment by i.p. injections and oral suspension.

FIG. 6 depicts, in accordance with an embodiment herein, EEG snapshots of before, during and after 3 days treatment. (A) depicts 3 pages of the before treatment, purposefully kept long in the interest of keeping the time scale the same for all snapshots (EEG duration 69 seconds/1 min 8 sec). (B) depicts during drug treatment containing a snapshot with 7 second seizure activity. (C) depicts after treatment snap shot 3 days post last treatment, with seizure duration of 16 seconds. Of note, before the treatment, the seizure activity was highly frequent with 20 to 30 or more seizure events per hour. During drug treatment, seizure frequency was found to be dramatically reduced.

FIG. 7 depicts, in accordance with an embodiment herein, a chart demonstrating application of creatine, phosphocreatine, or their analog with ethanol on GABAA receptor expressing cells abolish or dramatically (depending on concentration of creatine, phosphocreatine or their anaolog to ethanol concentration) reduce the positive allosteric effect of ethanol. Thus, in one embodiment, creatine, phosphocreatine and their analogs can be used to prevent and/or provide protection against alcohol addiction.

FIG. 8 depicts, in accordance with an embodiment herein, a chart demonstrating application of creatine, phosphocreatine, or their analog with ethanol on GABAA receptor expressing cells abolish or dramatically (depending on concentration of creatine, phosphocreatine or their analog to ethanol concentration) reduce the positive allosteric effect of ethanol. Thus, in one embodiment, creatine, phosphocreatine and their analogs can be used to prevent and/or provide protection against alcohol addiction.

FIG. 9 depicts, in accordance with an embodiment herein, a graph of results where 11 epileptic animals were treated initially with saline to establish base line then treated with twice daily injection of creatine at 30 mg/kg for 5 days. The data demonstrated significant efficacy in creation for treatment of epilepsy. Specifically, the figure depicts administration of cyclocreatine (100 nM).

FIG. 10 depicts, in accordance with an embodiment herein, a graph of results where 11 epileptic animals were treated initially with saline to establish base line then treated with twice daily injection of creatine at 30 mg/kg for 5 days. The data demonstrated significant efficacy in creation for treatment of epilepsy. Specifically, the figure depicts administration of phosphocreatine (100 nM).

FIG. 11 depicts, in accordance with an embodiment herein, a chart of data from additional epilepsy studies conducted by the inventors, where the results demonstrate a reduction in the number of seizures from administration of creatine.

FIG. 12 depicts, in accordance with an embodiment herein, a graph from additional epilepsy studies conducted by the inventors, where the results demonstrate a reduction in the number of seizures from administration of creatine as a percentage relative to control.

FIG. 13 depicts, in accordance with an embodiment herein, a chart of results of additional epilepsy studies conducted by the inventors, where the results demonstrate a reduction in seizure time from administration of creatine.

FIG. 14 depicts, in accordance with an embodiment herein, a graph from additional epilepsy studies conducted by the inventors, where the results demonstrate a reduction in percentage of total seizure time from administration of creatine relative to control.

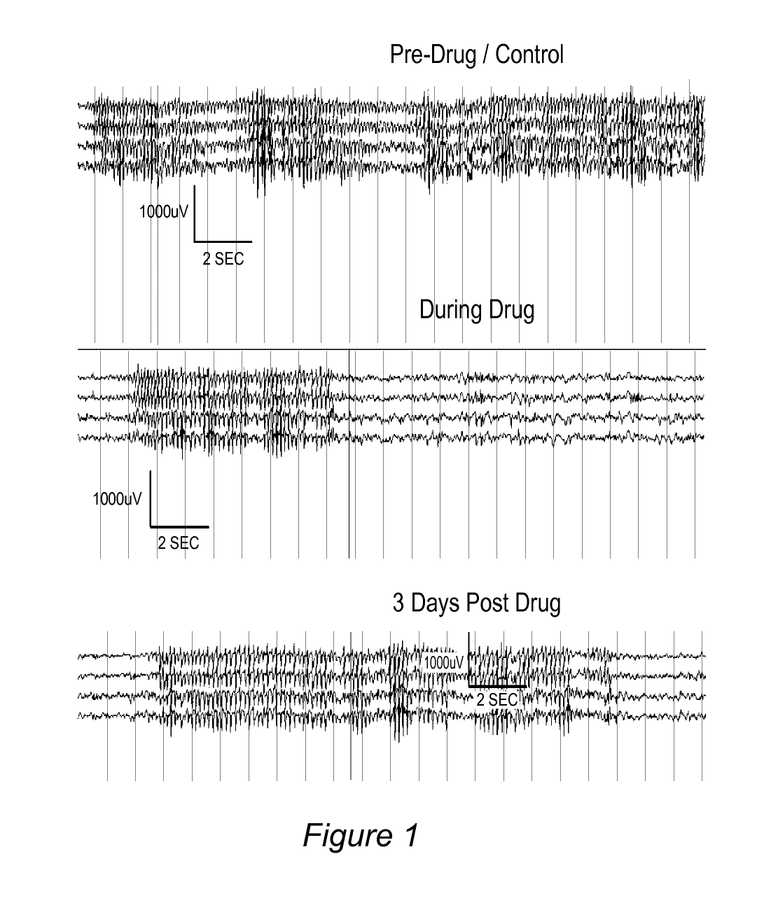

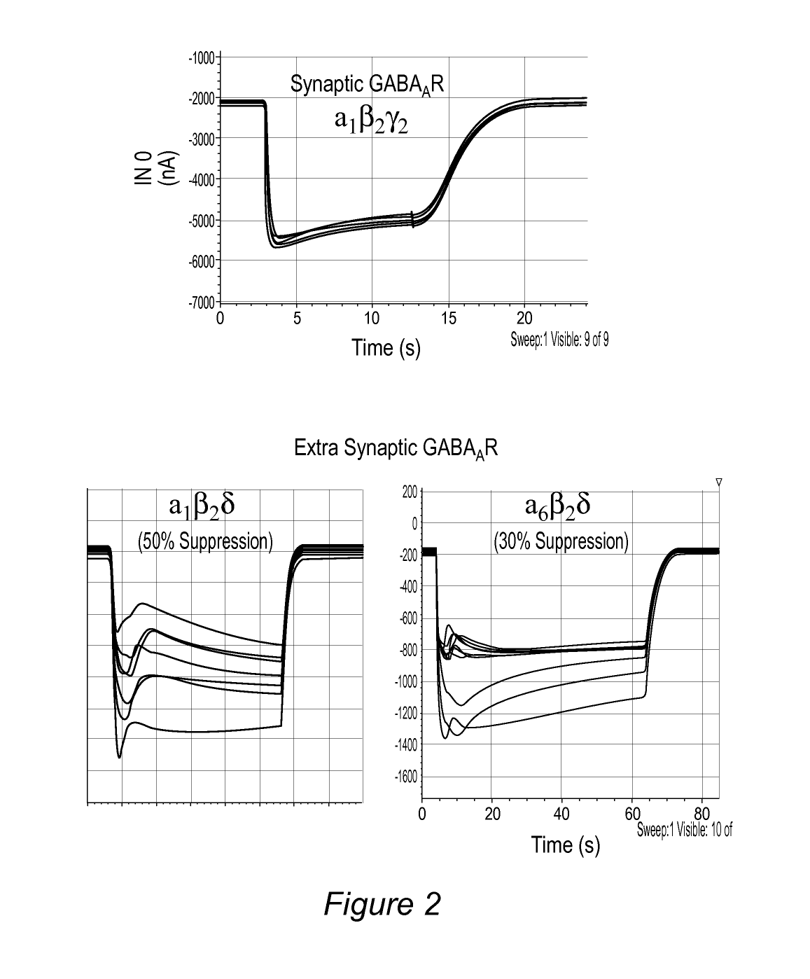

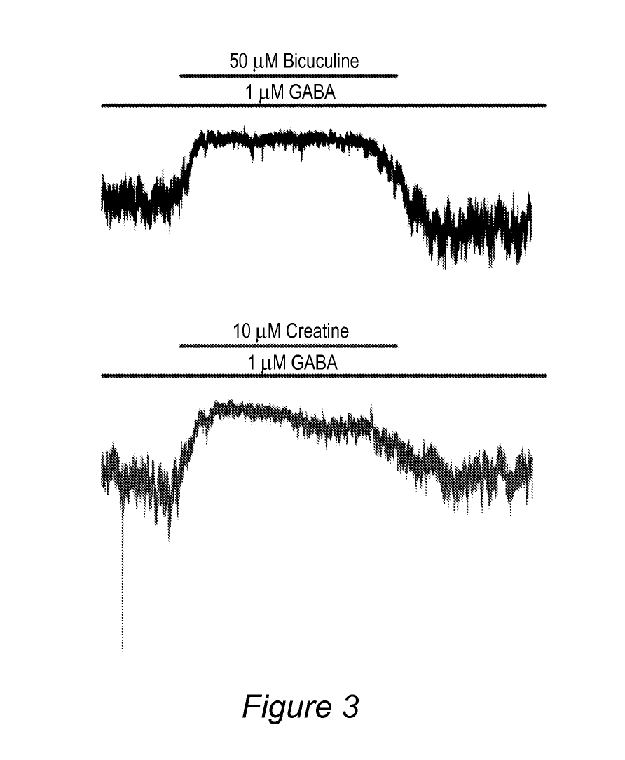

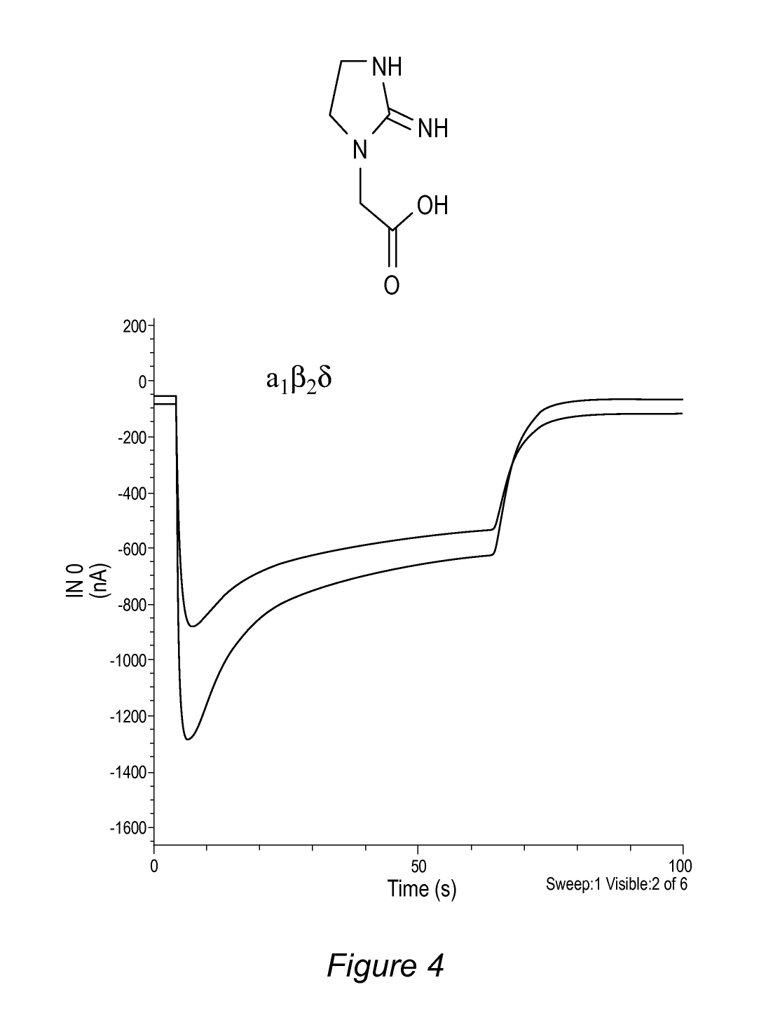

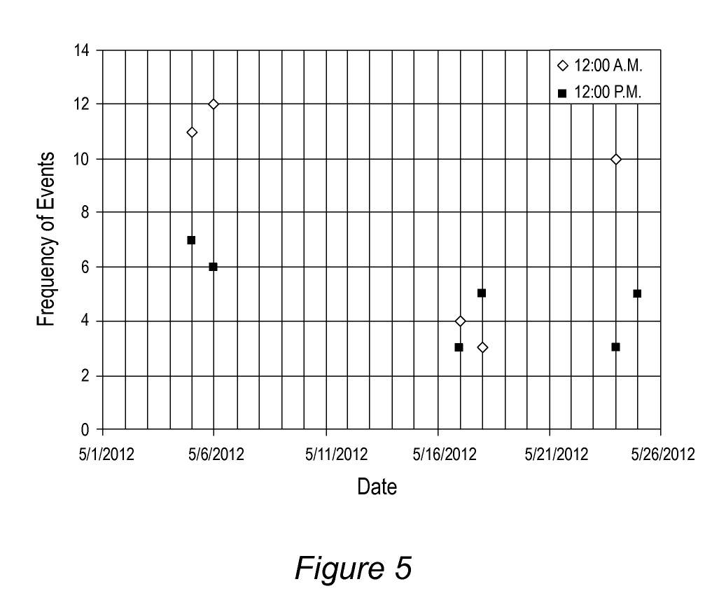

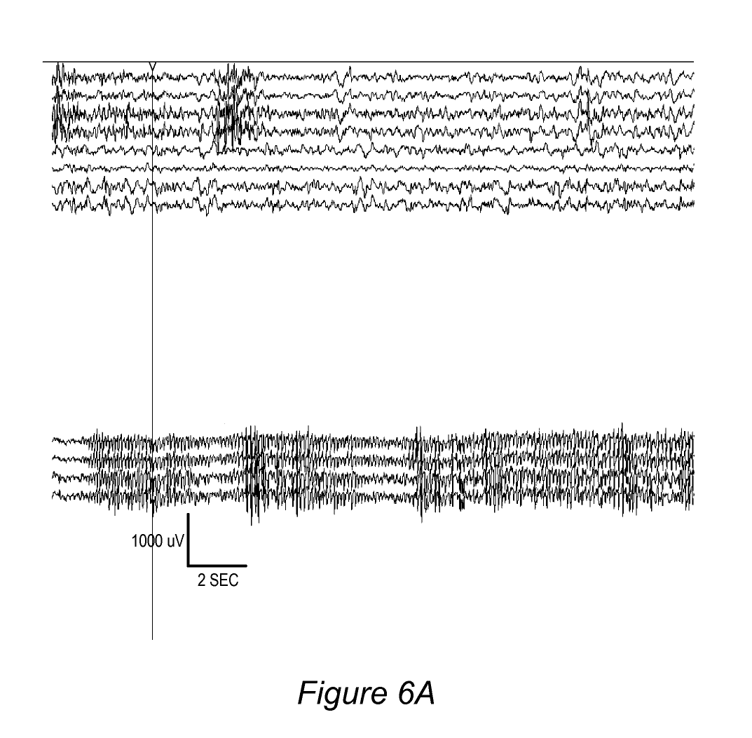

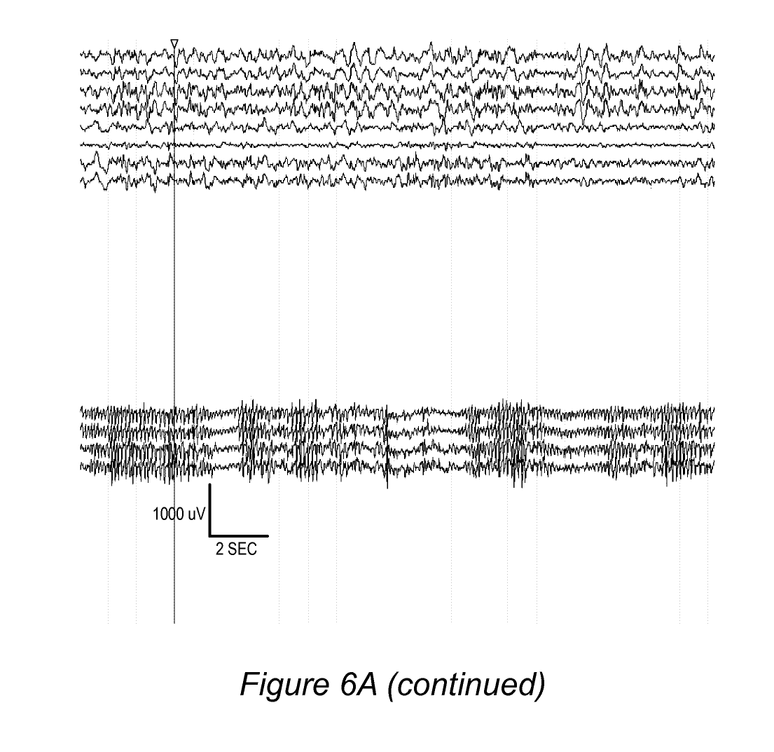

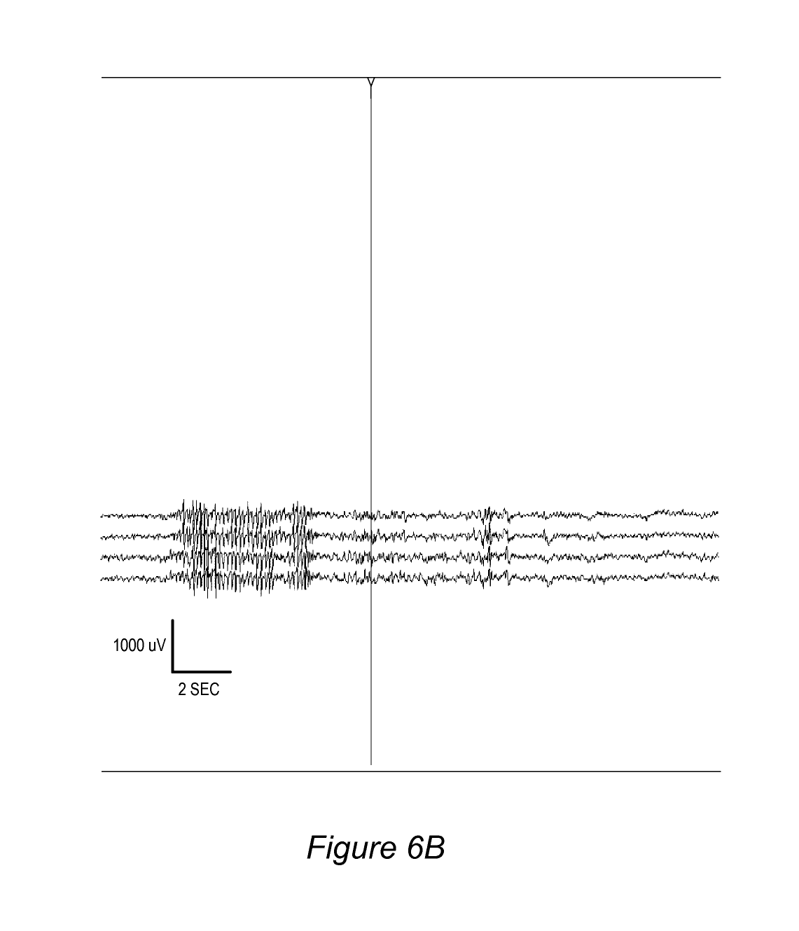

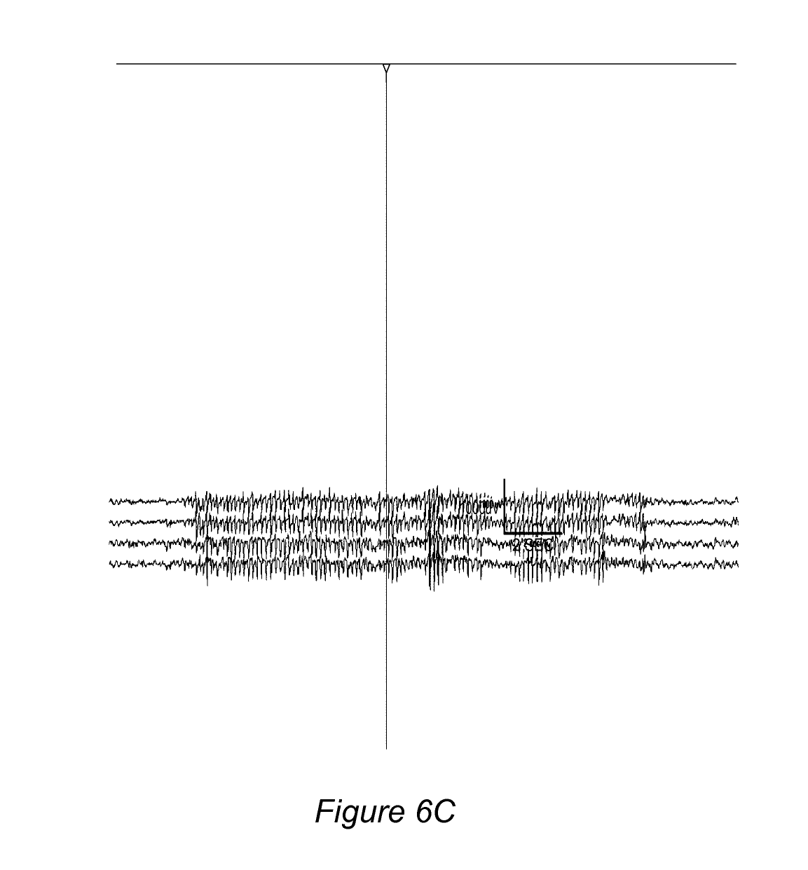

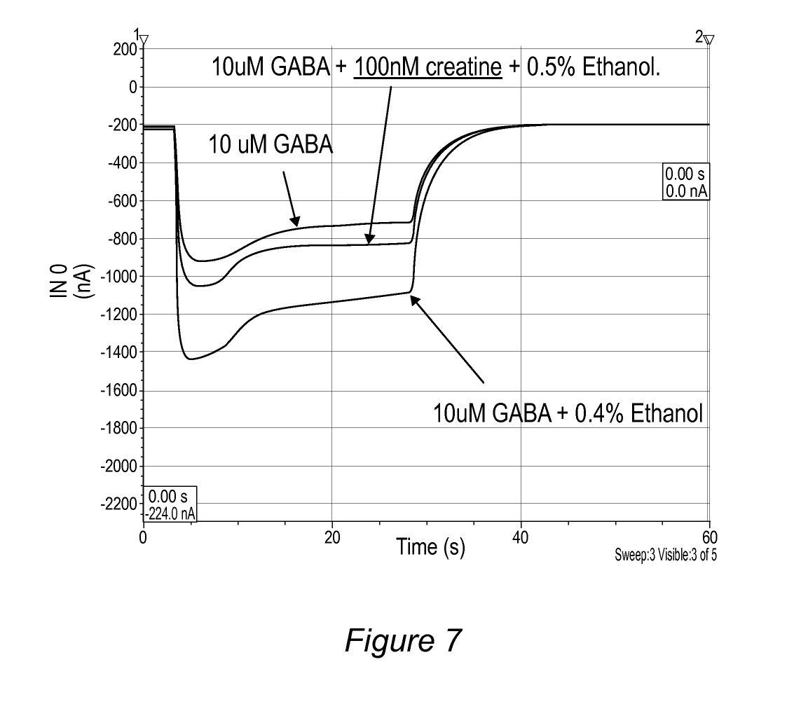

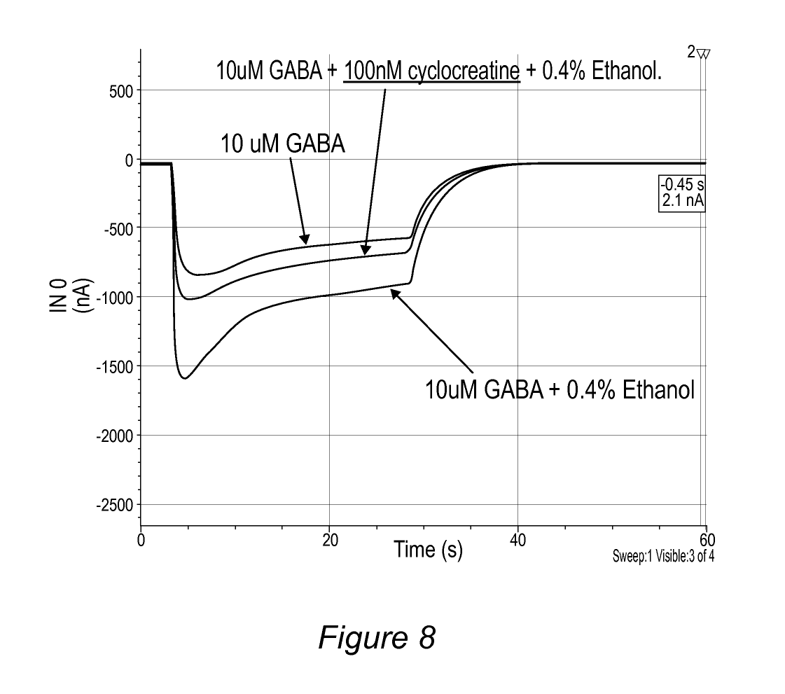

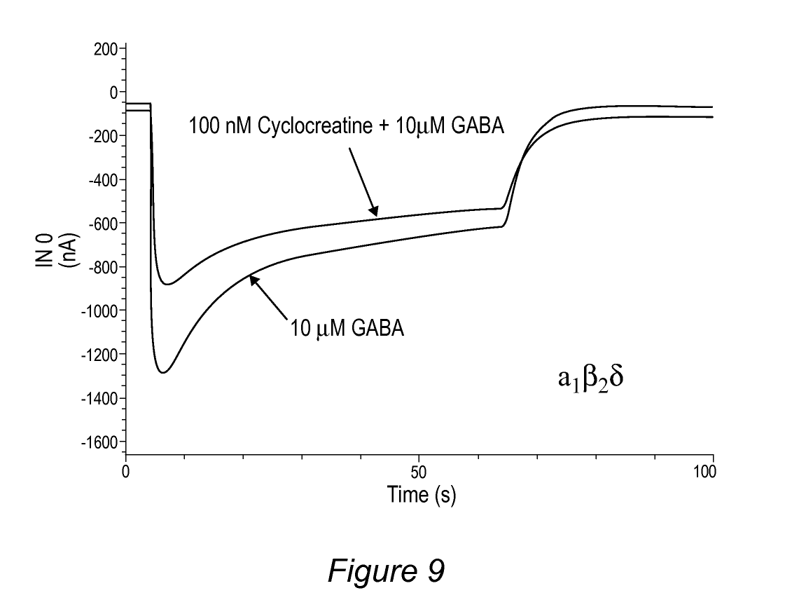

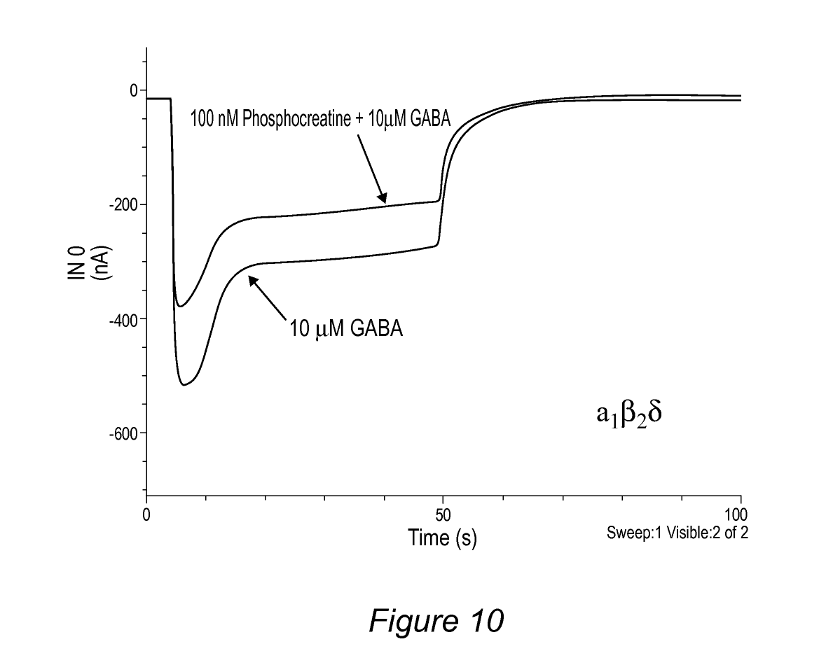

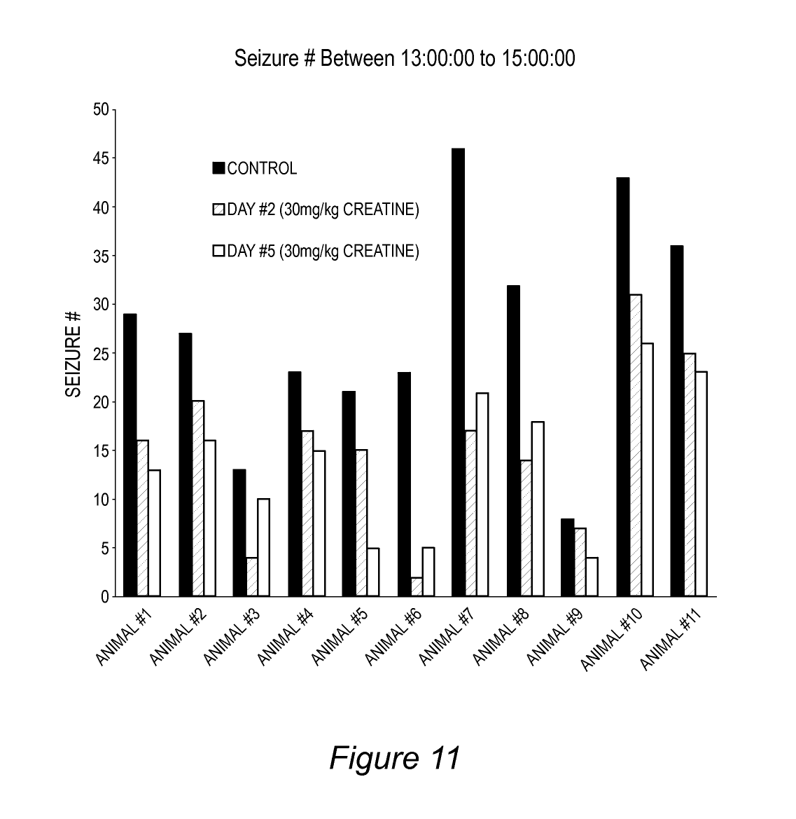

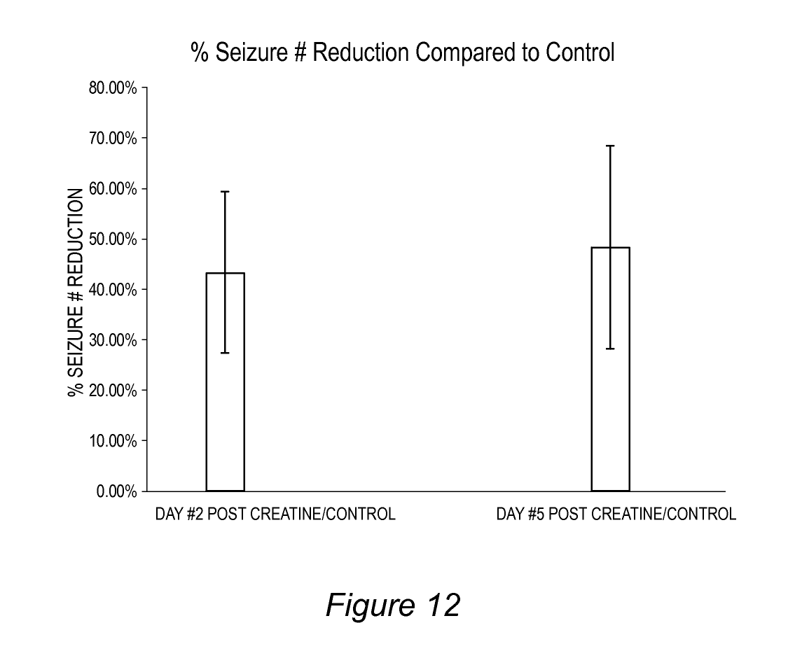

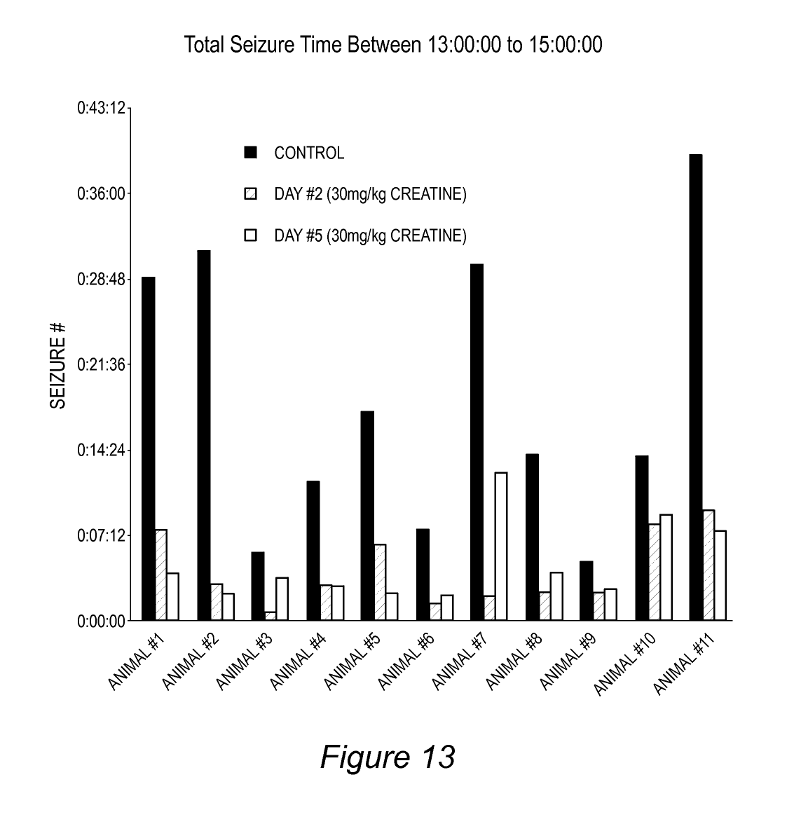

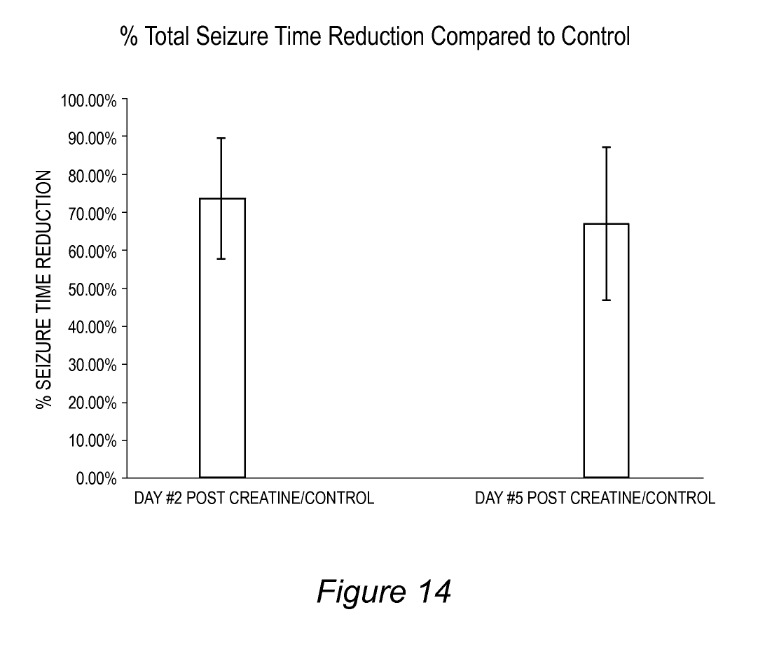

### DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3^{rd} ed., J. Wiley & Sons (New York, N.Y. 2001); March, Advanced Organic Chemistry Reactions, Mechanisms and Structure 5^{th} ed., J. Wiley & Sons (New York, N.Y. 2001); and Sambrook and Russel, Molecular Cloning: A Laboratory Manual 3rd ed., Cold Spring Harbor Laboratory Press (Cold Spring Harbor, N.Y. 2001), provide one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described.

As described herein, the inventors have developed an anti-epileptic treatment related to creatine and phosphocreatine. In one embodiment, the present invention provides a composition with anti-epileptic capability comprising a therapeutically effective dosage of creatine, phosphocreatine, and derivatives, analogs, pharmaceutical equivalent and/or salts thereof. In another embodiment, the composition is administered in combination with nutraceutical and/or anti-epileptic drugs. In another embodiment, the present invention may be combined with anti-epileptic drugs at lower dosages to provide fewer side effects and/or increased therapeutic efficacy. In another embodiment, the present invention may be combined with creatine supplements as a dietary regimen for epileptic patients.

As further disclosed herein, the inventors looked at the therapeutic potential of creatine for epileptic/seizure patients by giving creatine to rodents that have already developed epileptic seizures. The results demonstrate that creatine supplement to epileptic/seizure rats result in decrease in seizure frequency and duration. Specifically, experiments demonstrate that creatine, when given to epileptic/seizure patients can act therapeutically to diminish their suffering. In one embodiment, the present invention provides an alternative to individuals who are refractory to conventional antiepileptic drugs and, for example, have difficulty staying on a Ketogenic Diet. In another embodiment, the treatment may control seizure events in a patient in conjunction with administration of a ketogenic diet based. In another embodiment, the composition is administered in conjunction with a ketogenic diet, antiseizure medications, cyclocreatine and/or other creatine/phosphocreatine analogs.

In one embodiment, the present invention provides a method of treating epilepsy in a subject by administering a composition comprising a therapeutic effective dosage of creatine, phosphocreatine, and derivatives, analogs, pharmaceutical equivalent and/or salts thereof to the subject. In another embodiment, the epilepsy is treated by controlling seizures in the subject. In another embodiment, the composition is administered after the subject experiences a seizure event. In another embodiment, the composition decreases the seizure frequency in the subject. In another embodiment, the composition decreases the duration of the seizure in the subject. In another embodiment, the composition counteracts the seizure event itself in the subject. In another embodiment, the composition is administered 1 week or more after the subject experiences a seizure event. In another embodiment, the composition is administered between 1 week to 1 month after the subject experiences a seizure event. In another embodiment, the composition is administered 1 month or more after the subject experiences a seizure event. In one embodiment, the subject is a human. In another embodiment, the subject is a mouse or rodent. In another embodiment, the composition is administered orally. As apparent to one of skill in the art, the frequency of administration may be varied depending upon the size of the dosage. In one embodiment, the composition is administered once a day. In one embodiment, the composition is administered twice daily. In another embodiment, the composition is administered more than twice daily. In another embodiment, the composition is administered intravenously. As readily apparent to one of skill in the art, the dosage may be modified or varied depending upon the severity of the subject's condition. In one embodiment, the therapeutically effective dosage is about 30 mg/kg. In another embodiment, the therapeutically effective dosage is between 5 to 10 mg/kg. In another embodiment, the therapeutically effective dosage is between 10 to 20 mg/kg. In another embodiment, the therapeutically effective dosage is between 20 to 30 mg/kg. In another embodiment, the therapeutically effective dosage is between 30 to 40 mg/kg. In another embodiment, the therapeutically effective dosage is between 40 to 50 mg/kg. In another embodiment, the therapeutically effective dosage is between 50 to 80 mg/kg. In another embodiment, the therapeutically effective dosage is between 80 to 100 mg/kg. In another embodiment, the therapeutically effective dosage is between 100 to 500 mg/kg. In another embodiment, the therapeutically effective dosage is between 500 to 1000 mg/kg. In another embodiment, the therapeutically effective dosage is between 1000 to 1500 mg/kg.

As further disclosed herein and readily apparent to one of skill in the art, alcohol (Ethanol) can act as a positive allosteric modullator of extrasynaptic GABAA receptors. Previous studies have demonstrated that knockout or suppression of GABAA receptor in rodents result in prevention or significant reduction in alcohol addiction. In accordance with an embodiment herein, the present invention provides a treatment for alcoholism, where creatine is administered to a subject prior to drinking to suppress the effect alcohol have on GABA receptors, where some of the negative side effects of alcohol may be mitigated as well as help guard from developing alcoholism. In one embodiment, by administering creatine, the positive allosteric effect alcohol may have on GABA receptors is suppressed, and curtailing the conditioning effect of alcohol (positive allosteric effect) that result from continual exposure on GABA receptors. Continual alcohol use will always increase sensitivity of GABA receptors resulting in individuals' nervous system conditioned to the boost alcohol have on GABA receptor response. When alcohol is not present this boost is not present and result in the subject having an addiction craving. Thus, in one embodiment, creatine will mitigate the boosting effect of alcohol on extra-synaptic GABA receptor.

In one embodiment, the present invention provides a composition for treatment of alcohol addiction comprising a therapeutically effective dosage of creatine, phosphocreatine, and derivatives, analogs, pharmaceutical equivalent and/or salts thereof. In another embodiment, the composition is administered in combination with additional treatments for alcoholism. In one embodiment, the present invention provides a method of treating alcohol addiction in a subject by administering a composition comprising a therapeutic effective dosage of creatine, phosphocreatine, and derivatives, analogs, pharmaceutical equivalent and/or salts thereof to the subject. In one embodiment, the subject is a human. In another embodiment, the subject is a mouse or rodent. In another embodiment, the composition is administered orally. As apparent to one of skill in the art, the frequency of administration may be varied depending upon the size of the dosage. In one embodiment, the composition is administered once a day. In another embodiment, the composition is administered twice daily. In another embodiment, the composition is administered more than twice daily. In another embodiment, the composition is administered intravenously. In another embodiment, the composition is administered prior to drinking alcohol. In another embodiment, the composition is administered during drinking alcohol. As readily apparent to one of skill in the art, the dosage may be modified or varied depending upon the severity of the subject's condition. In one embodiment, the therapeutically effective dosage is about 30 mg/kg. In another embodiment, the therapeutically effective dosage is between 5 to 10 mg/kg. In another embodiment, the therapeutically effective dosage is between 10 to 20 mg/kg. In another embodiment, the therapeutically effective dosage is between 20 to 30 mg/kg. In another embodiment, the therapeutically effective dosage is between 30 to 40 mg/kg. In another embodiment, the therapeutically effective dosage is between 40 to 50 mg/kg. In another embodiment, the therapeutically effective dosage is between 50 to 80 mg/kg. In another embodiment, the therapeutically effective dosage is between 80 to 100 mg/kg. In another embodiment, the therapeutically effective dosage is between 100 to 500 mg/kg. In another embodiment, the therapeutically effective dosage is between 500 to 1000 mg/kg. In another embodiment, the therapeutically effective dosage is between 1000 to 1500 mg/kg.

As readily apparent to one of skill in the art, there are any number of disorders and/or diseases associated with seizures as well as GABA receptor signaling, and may be treated in conjunction with the various embodiments described herein. Thus, as readily apparent to one of skill in the art, the invention is in no way limited to treatment of epilepsy or alcohol addiction.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1

### Absence Epilepsy

### Petit Mal Seizures

Non-convulsive epileptic seizures resulting from abnormal electrical activity in the thalamo-cortical network. GABA positive allosteric modulators are contraindicative in treating absence epilepsy.

Typical Absence Seizures:
Paroxysmal loss of consciousness associated with bilaterally synchronous spike-and-wave discharges (SWD).
Seizure duration are brief, usually less than 20 seconds
Seizures onset are abrupt and sudden

Atypical Absence Seizures:
Do not suffer from loss of consciousness
Seizure onset and offset are more gradual
Frequency of seizure events are more frequent and longer
Associated with significant cognitive delays.
Refractory to medication with poor outcome.

### Example 2

### AY9944 Model of Atypical Absence Epilepsy

Induced via administering the cholesterol inhibitor AY9944 during postnatal developing period P2 to P21.
Epileptic seizure is chronic, resembling all the symptoms of atypical absence epilepsy
   Failure to show ictal immobility
   Slow spike-wave discharges
   Cognitive impairments
   Counterintuitive with application of GABA Positive allosteric modulators and Agonists.
   Greater seizure susceptibility in female than male.

### Example 3

### Creatine's Therapeutic Action Through GABA Receptors

Increase in CNS GABA levels cause SWD similar to absence seizures.
In the cholesterol synthesis inhibition (CSI) model, neuronal sensitivity to Zn⁺² is significantly increased. Similarly, there is a loss of sensitivity to benzodiazepine. This suggests a decrease in γ2 subunit expression.
In CSI rodent model, α1 and γ2 subunit mRNA were found to be lower in the thalamus but higher in the cortex compared to control
During CSI drug treatment and up to 6 days post drug treatment, 17 to 50% drop in GABA_{A} and GABA_{B} receptor proteins found in lipid rafts.
Like clinical absence epilepsy, application of positive GABA allosteric modulators and GABA agonists resulted in exacerbation of seizure events

### Example 4

### Generally

The inventors found creatine to be effective in reducing both frequency and duration of epileptic seizures in atypical absence epilepsy rodent models.
Creatine act as a partial negative allosteric modulator of extra-synaptic GABA_{A} receptors.
The creatine analog cyclocreatine does not confer the same therapeutic effect as creatine, indicating that creatine therapeutic effect cannot be fully attributed to its extra-synaptic GABA effect

### Example 5

### Creatine and Phosphocreatine

### Antiepileptic Drugs

Creatine and/or phosphocreatine can be used as an antiepileptic drug. Administering creatine to epileptic rodent model resulted in a dramatic (greater than 70%) reduction in both frequency and duration of epileptic seizure events. In addition, the inventors' data show that compared to current antiepileptic drugs, a single dose of creatine last much longer than conventional antiepileptic drugs. Investigation into phosphocreatine on expression cells indicate it to have the same effect as creatine.

The creatine and/or phosphocreatine can be used by itself or in conjuncture with existing antiepileptic drugs in the treatment of epilepsy. Additionally, creatine and/or phosphocreatine supplement can be used in place of the Ketogenic diet for adults and in combination with the Ketogenic diet for children. Ketogenic diet is a diet prescribed by doctors to epileptic patients, but due to its rigorous dietary regiment of high fat and low carb, only children can stay on this diet. The ketogenic diet is extremely difficult for adults to follow.

### Example 6

### Advantages

Creatine and phosphocreatine is safe to administer, and does not cause many of the numerous side effects caused by current antiepileptic drugs. Creatine has been administered at high dosage to neurodegenerative disease patients and taken by athletes with few reported side effects. Additionally, as described herein, the inventors have demonstrated that creatine alone will dramatically reduce the frequency and duration of epileptic seizures in the rodent epilepsy model. In addition, creatine appears to have longer efficacy duration than conventional drugs. Data from expression cells demonstrate similar effect with phosphocreatine. Also, creatine and phosphocreatine supplement can be used as an alternative for the hard to follow Ketogenic diet for adults. For children creatine supplement can complement the Ketogenic diet. The Ketogenic diet works only for some of the patients, and the creatine complement can increase the number of positive patient outcomes. Finally, creatine and phosphocreatine do not pose many of the toxic/negative side effects such as hepatoxicity, depression, suicidal thoughts, and infertility associated with current antiepileptic drugs.

### Example 7

### Treatment of Alcohol Addiction Through Use of Creatine, Phosphocreatine, and Derivatives, Analogs, Pharmaceutical Equivalents and/or Salts Thereof

Alcoholism is a serious problem both to the individual, the individual's family, and society. Currently there is no compound that can effectively prevent alcoholism. Alcohol (Ethanol) acts as a positive allosteric modulator of extrasynaptic GABAA receptors. Previous studies have demonstrated that knockout or suppression of GABAA receptor in rodents result in prevention or significant reduction in alcohol addiction. The inventors have found that creatine, phosphocreatine and their analogs are effective in preventing or significantly diminishing the GABAA receptor positive allosteric modulator effect of alcohol (ethanol). As further described herein, the inventors have demonstrated that co-application of creatine, phosphocreatine, or their analog with ethanol on GABAA receptor expressing cells abolish or dramatically (depending on concentration of creatine, phosphocreatine or their anaolog to ethanol concentration) reduce the positive allosteric effect of ethanol. Thus creatine, phosphocreatine and their analogs may be used to prevent and/or provide protection against alcohol addiction. Creatine can be used to supplement alcoholic drinks to significantly reduce the societal level of alcohol addiction.

### Example 8

### Additional Epilepsy Studies

11 epileptic animals were treated initially with saline to establish base line (day 0), then treated with twice daily injection of creatine at 30 mg/kg (days 1-5). The data demonstrated significant efficacy in creation for treatment of epilepsy. Specifically, the inventors performed the following:
Day 0—Control (saline injection)
Injection of creatine between days 1-5—twice daily injection of 30 mg/kg of creatine
Day #2 (30 mg/kg creatine)
Day #5 (30 mg/kg creatine)

### Example 9

### Additional Epilepsy Studies

### Seizure Number Reduction

TABLE 1
Seizure # Between 13:00:00 to 15:00:00

| | Day 0 | Day 2 | Day 5 |
|---|---|---|---|
| Animal #1 | 29 | 16 | 13 |
| Animal #2 | 27 | 20 | 16 |
| Animal #3 | 13 | 4 | 10 |
| Animal #4 | 23 | 17 | 15 |
| Animal #5 | 21 | 15 | 5 |
| Animal #6 | 23 | 2 | 5 |
| Animal #7 | 46 | 17 | 21 |
| Animal #8 | 32 | 14 | 18 |
| Animal #9 | 8 | 7 | 4 |
| Animal #10 | 43 | 31 | 26 |
| Animal #11 | 36 | 25 | 23 |

TABLE 2
% Seizure # Reduction

| | Day #2 Post Creatine/ | Day #5 Post Creatine/ |
|---|---|---|
| | Control | Control |
| Animal #1 | 44.83% | 55.17% |
| Animal #2 | 25.93% | 40.74% |
| Animal #3 | 69.23% | 23.08% |
| Animal #4 | 26.09% | 34.78% |
| Animal #5 | 28.57% | 76.19% |
| Animal #6 | 91.30% | 78.26% |
| Animal #7 | 63.04% | 54.35% |
| Animal #8 | 56.25% | 43.75% |
| Animal #9 | 12.50% | 50.00% |
| Animal #10 | 27.91% | 39.53% |
| Animal #11 | 30.56% | 36.11% |
| Average = | 43.29% | 48.36% |
| StDev = | 23.87% | 16.98% |

### Example 10

### Additional Epilepsy Studies

### Seizure Time Reduction

TABLE 3
Total Seizure Time Between 13:00:00 to 15:00:00

| | Sep. 13, 2012 | Sep. 15, 2012 | Sep. 18, 2012 |
|---|---|---|---|
| Animal #1 | 0:29:02 | 0:07:43 | 0:04:04 |
| Animal #2 | 0:31:11 | 0:03:08 | 0:02:23 |
| Animal #3 | 0:05:51 | 0:00:50 | 0:03:42 |
| Animal #4 | 0:11:49 | 0:03:04 | 0:03:00 |
| Animal #5 | 0:17:44 | 0:06:31 | 0:02:25 |
| Animal #6 | 0:07:42 | 0:01:28 | 0:02:16 |
| Animal #7 | 0:30:05 | 0:02:11 | 0:12:34 |
| Animal #8 | 0:14:08 | 0:02:30 | 0:04:10 |
| Animal #9 | 0:05:05 | 0:02:26 | 0:02:44 |
| Animal #10 | 0:13:57 | 0:08:15 | 0:08:58 |
| Animal #11 | 0:39:18 | 0:09:24 | 0:07:38 |

TABLE 4
% Total Seizure Time Reduction

| | Day #2 Post Creatine/ | Day #5 Post Creatine/ |
|---|---|---|
| | Control | Control |
| Animal #1 | 73.42% | 85.99% |
| Animal #2 | 89.95% | 92.36% |
| Animal #3 | 85.75% | 36.75% |
| Animal #4 | 74.05% | 74.61% |
| Animal #5 | 63.25% | 86.37% |
| Animal #6 | 80.95% | 70.56% |
| Animal #7 | 92.74% | 58.23% |
| Animal #8 | 82.31% | 70.52% |
| Animal #9 | 52.13% | 46.23% |
| Animal #10 | 40.86% | 35.72% |
| Animal #11 | 76.08% | 80.58% |
| Average = | 73.77% | 67.08% |
| StDev = | 15.99% | 20.12% |

The various methods and techniques described above provide a number of ways to carry out the invention. Of course, it is to be understood that not necessarily all objectives or advantages described may be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as may be taught or suggested herein. A variety of advantageous and disadvantageous alternatives are mentioned herein. It is to be understood that some preferred embodiments specifically include one, another, or several advantageous features, while others specifically exclude one, another, or several disadvantageous features, while still others specifically mitigate a present disadvantageous feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be mixed and matched by one of ordinary skill in this art to perform methods in accordance with principles described herein. Among the various elements, features, and steps some will be specifically included and others specifically excluded in diverse embodiments.

Although the invention has been disclosed in the context of certain embodiments and examples, it will be understood by those skilled in the art that the embodiments of the invention extend beyond the specifically disclosed embodiments to other alternative embodiments and/or uses and modifications and equivalents thereof.

Many variations and alternative elements have been disclosed in embodiments of the present invention. Still further variations and alternate elements will be apparent to one of skill in the art. Among these variations, without limitation, are the selection of constituent modules for the inventive compositions, and the diseases and other clinical conditions that may be diagnosed, prognosed or treated therewith. Various embodiments of the invention can specifically include or exclude any of these variations or elements.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

In some embodiments, the terms “a” and “an” and “the” and similar references used in the context of describing a particular embodiment of the invention (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Preferred embodiments of this invention are described herein, including the best mode known to the inventors for carrying out the invention. Variations on those preferred embodiments will become apparent to those of ordinary skill in the art upon reading the foregoing description. It is contemplated that skilled artisans can employ such variations as appropriate, and the invention can be practiced otherwise than specifically described herein. Accordingly, many embodiments of this invention include all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above cited references and printed publications are herein individually incorporated by reference in their entirety.

In closing, it is to be understood that the embodiments of the invention disclosed herein are illustrative of the principles of the present invention. Other modifications that can be employed can be within the scope of the invention. Thus, by way of example, but not of limitation, alternative configurations of the present invention can be utilized in accordance with the teachings herein. Accordingly, embodiments of the present invention are not limited to that precisely as shown and described.

1. A method of treating epileptic seizures in an subject, comprising:
providing a composition comprising a therapeutically effective dosage of creatine, cyclocreatine, phosphocreatine, or a derivative, analog, salt, and/or pharmaceutical equivalent thereof; and
administering the composition to the subject.

2. The method of claim 1, wherein the therapeutically effective dosage is between 5 mg/kg to 15 mg/kg.

3. The method of claim 1, wherein the therapeutically effective dosage is between 15 mg/kg to 50 mg/kg.

4. The method of claim 1, wherein the therapeutically effective dosage is between 50 mg/kg to 100 mg/kg.

5. The method of claim 1, wherein the therapeutically effective dosage is between 100 mg/kg to 500 mg/kg.

6. The method of claim 1, wherein the therapeutically effective dosage is between 500 mg/kg to 1000 mg/kg.

7. The method of claim 1, wherein the therapeutically effective dosage is between 1000 mg/kg and 1500 mg/kg.

8. The method of claim 1, wherein the composition is administered twice daily.

9. The method of claim 1, wherein the composition is administered once a day.

10. The method of claim 1, wherein the composition is administered orally.

11. The method of claim 1, wherein the subject is a human.

12. The method of claim 1, wherein the subject is a rodent.

13. The method of claim 1, wherein the subject has previously experienced an epileptic seizure.

14. The method of claim 1, wherein the subject has previously experienced an epileptic seizure less than 3 days before treatment.

15. The method of claim 1, wherein the subject has previously experienced an epileptic seizure between 3 to 7 days before treatment.

16. The method of claim 1, wherein the subject has previously experienced an epileptic seizure between 7 days to 30 days before treatment.

17. The method of claim 1, wherein the subject has previously experienced an epileptic seizure more than 30 days before treatment.

18. The method of claim 1, wherein the composition is administered in conjunction with additional seizure treatments.

19. The method of claim 1, wherein administering the composition results in a decrease in frequency of epileptic seizures in the subject.

20. The method of claim 1, wherein administering the composition results in a decrease in duration of epileptic seizures in the subject.

21. A method of treating alcohol addiction in a subject, comprising:
providing a composition comprising a therapeutically effective dosage of creatine, cyclocreatine, phosphocreatine, or a derivative, analog, salt, and/or pharmaceutical equivalent thereof; and
administering the composition to the subject.

22. The method of claim 21, wherein the therapeutically effective dosage is between 5 mg/kg to 15 mg/kg.

23. The method of claim 21, wherein the therapeutically effective dosage is between 15 mg/kg to 50 mg/kg.

24. The method of claim 21, wherein the therapeutically effective dosage is between 50 mg/kg to 100 mg/kg.

25. The method of claim 21, wherein the therapeutically effective dosage is between 100 mg/kg to 500 mg/kg.

26. The method of claim 21, wherein the therapeutically effective dosage is between 500 mg/kg to 1000 mg/kg.

27. The method of claim 21, wherein the therapeutically effective dosage is between 1000 mg/kg and 1500 mg/kg.

28. The method of claim 21, wherein the composition is administered twice daily.

29. The method of claim 21, wherein the composition is administered once a day.

30. The method of claim 21, wherein the composition is administered orally.

31. The method of claim 21, wherein the subject is a human.

32. The method of claim 21, wherein the subject is a rodent.

33. A pharmaceutical composition, comprising:
a therapeutically effective dosage of creatine, cyclocreatine, phosphocreatine, or a derivative, analog, salt, and/or pharmaceutical equivalent thereof; and
a pharmaceutically acceptable carrier.

34. The pharmaceutical composition of claim 33, wherein the therapeutically effective dosage is about 30 mg/kg.

35. The pharmaceutical composition of claim 33, wherein the therapeutically effective dosage is about 100 mg/kg.

36. The pharmaceutical composition of claim 33, wherein the therapeutically effective dosage is about 1000 mg/kg.
